(19) United States
(12) Patent Application Publication
(10) Pub. No.: US 2004/0265357 A1
(43) Pub. Date: Dec. 30, 2004

(54) Combination of liponic acid and glutamine in food and pharmaceutical products

(76) Inventors: Klause Kramer, Landau (DE); Erich Roth, Altenberg (AT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/492,839

(22) PCT Filed: Oct. 18, 2002
(86) PCT No.: PCT/EP02/11692

(30) Foreign Application Priority Data
Oct. 19, 2001 (DE) 101 51 764.5

(51) Int. Cl. A61K 31/385; A61K 31/198; A61K 47/00

(52) U.S. Cl. 424/439; 514/440; 514/563

(57) ABSTRACT

The present invention relates to the use of at least one lipoic acid, of a physiologically acceptable derivative or salt thereof; and of at least one glutamine, of a physiologically acceptable derivative or salt thereof, for nutritional supplementation, in functional foods and for therapeutic purposes, namely in particular for the treatment of particular disorders associated with a glutathione deficiency. Also described are compositions having a corresponding active ingredient combination, and compositions in the form of commercial packs with corresponding combination products or single-component products for combined use.

The present invention relates to the use of lipoic acid and glutamine for food supplementation, in functional foods and for therapeutic purposes, to compositions having a corresponding active ingredient combination, and to compositions in the form of commercial packs with corresponding combination products or single-component products for combined use.

Lipoic acid is a coenzyme in the oxidative decarboxylation of α-keto acids and is found in virtually every cell in the body. The antiinflammatory, analgesic and cytoprotective properties of lipoic acid, and its antioxidant effect, make it an interesting ingredient for pharmacy, cosmetics, food science and adjacent areas (Biothiols in Health and Disease, edited by Packer L. and Cadenas E., Marcel Dekker Inc., New York, Basle, Hong Kong; Biwenga GP, et al., Gen Pharmac 29:3:315-331; 1997; Packer L, et al., Free Rad Biol Med 19:2:227-250; 1995). Thus, various studies on diabetic patients in which administration of lipoic acid showed an effect have been reported (e.g. Jacob et al., in Free Radical Biology & Medicine, Vol. 27, Nos. 3/4 (1999) 309-314, and BioFactors 10 (1999) 169-174). Moreover, Stoll et al. reported in Pharmacology Biochemistry and Behavior, Vol. 46, pp. 799-805 (1993) and in Ann. NY Acad. Sci., Vol. 717, pp. 122-128 (1994), respectively, that lipoic acid is able to improve the long-term memory of aged mice and the cognitive abilities of rodents. Han D. et al. postulate, in American Journal of Physiology 273: R 1771-1778 (1997) a lipoic acid-mediated protection against glutamate-induced depletion of intracellular glutathione and use this in an attempt to give a mechanistic explanation of the neuroprotective effects of lipoic acid which are observed in rat ischemia models. In experimentally induced cerebral ischemia, lipoic acid (iv, 25 mg/kg body weight) and dihydrolipoic acid (50 and 100 mg/kg body weight) brought about a reduction in the infarct size (Panigrahi M, et al., Brain Res 717:184-188; 1996). Hagen T. M. et al., in FASEB J. 13, 411-418 (1999), observed an improvement in mitochondrial function in rats after oral administration of (R)-α-lipoic acid. Subcutaneous injection of lipoic acid (25 mg/kg body weight) twice led to a significant reduction in cataract induced by buthionine sulfoximine in an experimental rat model (Maitra I, et al., Free Rad Biol Med 18:4:823-829; 1995). Not only the R enantiomer and the racemic mixture are said to be effective in this case, but also the S enantiomer (Maitra I, et al., Biochem Biophys Res Comms 221:422-429; 1996). It has likewise been shown in a model of diabetes that the lens damage induced by diabetes, and changes in the redox status were partially prevented (Obrosova I, et al., Diabetologia 41:1442-1450; 1998). Borcea V, et al. reported (Free Rad Bio Med 22:11/12:1495-1500; 1999) a reduction in oxidative stress in diabetes patients on daily administration of 600 mg of lipoic acid for more than 3 months. It was further shown that intake of lipoic acid prevents the symptoms of vitamin E deficiency phenomena (muscular dystrophy, weight loss) when mice 6-8 weeks old received in addition to a vitamin E-deficient diet 1.65 g of lipoic acid/kg of body weight (Podda M, et al., Biochem Biophys Res Comms 204:98-104; 1994).

In Germany, the lipoic acid-containing products are currently listed for the treatment of abnormal sensation associated with diabetic polyneuropathy. Formulations of solid salts of lipoic acid are proposed in U.S. Pat. No. 5,990,152. U.S. Pat. No. 5,994,393 relates to a further modification of lipoic acid. Useful lipoic acid analogs are proposed in WO 99/45922. Combinations of lipoic acid and vitamins for producing drugs are described in EP 0 572 922 A1.

Glutamine is the quantitatively most important free amino acid in the human body (cf. Roth E, et al., Wien Klin Wochenschr 108:669-676). Whereas glutamine is absolutely essential for the growth of cells in cell culture, this essentiality does not exist in the human body because glutamine is synthesized there from other, likewise essential amino acids. Skeletal muscle is the principal glutamine-synthesizing organ. Glutamine is released from skeletal muscle not only in the fasting state or in the condition of clinical protein catabolism but also in the postprandial state. Organs which take up glutamine are in particular the intestine, the immune cells, the kidney (for buffering acidic valencies) and, depending on the physiological condition, the liver. Glutamine appears to be important in particular for rapidly proliferating cells such as mucosal cells and immune cells. It has recently been pointed out that glutamine is not only important as nitrogen donor and protein constituent but may also have a cell-regulating power similar to a hormone or a cytokine. Thus, glutamine stimulates the expression of surface antigens on monocytes and lymphocytes, influences the formation of cytokines, the synthesis of stress proteins (heat shock proteins) and the glutathione level (Hong R W, et al., Ann Surg 213:114-119; 1992; Yu J. C., et al., Clin Nutr 15:261-265; 1996; Denno R, et al., J Surg Res 61:35-38; 1996). In addition, glutamine is able to influence the cell cycle arrest from G0 to G1 and to reduce the ability of carcinoma cells to undergo apoptosis. Further investigations have revealed that oral administration of glutamine to mice resulted in an increased number of lymphocytes in Peyer's patches (Manhart N, et al., Clin Nutr 19:197-201; 2000).

It has now been found that certain combined uses of lipoic acids and glutamines open up a surprisingly effective possibility of treating a large number of disorders associated with glutathione deficiency and metabolic deficits associated therewith, and thus represent an ideal nutritional supplementation, confer on food products a beneficial effect on health, and have a high therapeutic value.

The present invention therefore relates to the use of at least one lipoic acid, physiologically acceptable derivative or salts thereof and at least one glutamine, physiologically acceptable derivatives or salts thereof for nutritional supplementation, in functional foods and for therapeutic purposes.

The use according to the invention represents a combined use, i.e. the use of at least one lipoic acid, physiologically acceptable derivatives or salts thereof—also referred to hereinafter as “lipoic acid component” for simplicity—and the use of at least one glutamine, physiologically acceptable derivatives or salts thereof—also referred to hereinafter as glutamine component for simplicity—takes place in a context appropriate for the purpose, in particular with a view to optimal activity. Thus, the lipoic acid component and the glutamine component can in principle be administered together in one formulation or separately in at least two different formulations. Administration of separate formulations includes both concurrent administration, i.e. taking place at essentially the same times or in direct succession, and sequential administration, i.e. taking place at different times. A particular embodiment of sequential administration is achieved by alternating administration of the two components, for example with an early/late daily rhythm. Concurrent administration is preferred.

Thus, the present invention relates both to the use of at least one lipoic acid, physiologically acceptable derivatives or salts thereof and to the use of at least one glutamine, physiologically acceptable derivatives or salts thereof for glutamine- or lipoic acid-assisted use. In this sense, the invention relates to compositions which are based on a combination i) of at least one lipoic acid, physiologically acceptable derivatives or salts thereof; and ii) of at least one glutamine, physiologically acceptable derivatives or salts thereof, and, where appropriate, other active ingredients, it being possible for the active ingredient components, in particular components i) and ii), to be formulated together or separately.

The term “lipoic acid” refers according to the invention to 5-(1,2-dithiolan-3-yl)valeric acid, also called thioctic acid, of the formula I

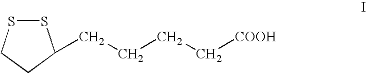

including the optical isomers covered by this formula, both as mixtures, e.g. racemates, and in pure form, e.g. R or S enantiomers. The preferred isomer is (R)-5-(1,2-dithiolan-3-yl) valeric acid of the formula II

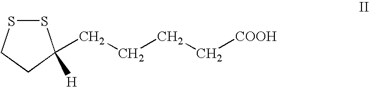

Lipoic acid mixtures with an (R) enantiomeric excess (ee) of at least 40% are preferred. The (R) enantiomeric excess is preferably at least 80%, in particular at least 98%.

The enantiomeric excess (ee) results in this connection from the following formula: (ee)[%]=(R−S)/(R+S)×100. R and S are the descriptors of the CIP system for the two enantiomers and reflect the absolute configuration at the asymmetric C(5) atom. The enantiopure compound (ee=100%) is also referred to as homochiral compound.

Lipoic acid derivatives include, in particular, synthesis precursors and metabolites of lipoic acid, i.e. especially dihydrolipoic acid. Further metabolites which should be mentioned are lipoamide, lipoyllysine, di-6,8-bisnorlipoic acid and tetranorlipoic acid. Further suitable lipoic acid derivatives are the esters, thioesters and amides of lipoic acid with amino alcohols, amino thiols and diamines which are described in WO 99/45922 as lipoic acid analogs of the formula (I) and which are incorporated in the present application by reference. Corresponding to the statements about lipoic acid, the respective optical isomers of the derivatives also belong therewith.

The physiologically acceptable salts of lipoic acid also lipoic acid derivatives are in the present case preferably base addition salts.

The base addition salts include salts with inorganic bases, for example metal hydroxides or carbonates of alkali metals, alkaline earth metals or transition metals, or with organic bases, for example ammonia or basic amino acids such as arginine and lysine, amines, e.g. methylamine, dimethylamine, trimethylamine, triethylamine, ethylamine, diethylamine, ethylenediamine, ethanolamine, diethanolamine, 1-amino-2-propanol, 3-amino-1-propanol or hexamethylenetetramine, saturated cyclic amines having 4 to 6 ring carbon atoms, such as piperidine, piperazine, pyrrolidine and morpholine, and other organic bases, for example N-methylglucamine, creatine, trometamol and tromethamine, and quaternary ammonium compounds such as tetramethylammonium and the like.

Salts with inorganic bases, e.g. Na, K, Mg, Ca, Zn, Cr and Fe salts, and for the area of enteral processing the trometamol salt, are preferred.

The term “glutamine” refers according to the invention to 2-aminopentanoic acid 5-amide of the formula III

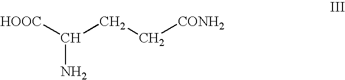

including the optical isomers covered by this formula, both as mixtures, e.g. racemates, and in pure form, e.g. R or S enantiomers. The preferred isomer is (S)-2-aminopentanoic acid 5-amide, also referred to as L-glutamine, of the formula IV

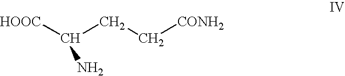

Glutamine mixtures with an (S) enantiomeric excess (ee) of at least 60% are preferred. The (S) enantiomeric excess is preferably at least 90.0%, in particular at least 99.9%.

Glutamine derivatives include, in particular, synthesis precursors and metabolites of glutamine, i.e. especially dipeptides, in particular L-alanyl-L-glutamine and L-glycyl-L-glutamine. Corresponding to the statements about glutamine, the respective optical isomers of the derivatives also belong therewith.

The physiologically acceptable salts of glutamines or glutamine derivatives include in the present case both base and acid addition salts.

The base addition salts include salts of glutamines or glutamine derivatives with the bases mentioned above in connection with lipoic acid and lipoic acid derivatives.

The acid addition salts include salts of glutamines or glutamine derivatives with inorganic acids such as hydrochloric acid, sulfuric acid, nitric acid or phosphoric acid, or organic acids, in particular carboxylic acids, e.g. acetic acid, tartaric acid, lactic acid, citric acid, malic acid, mandelic acid, ascorbic acid, maleic acid, fumaric acid, gluconic acid or sulfonic acids, e.g. methanesulfonic acid, benzenesulfonic acid and toluenesulfonic acid, and the like.

Besides the lipoic acid and glutamine components, the use according to the invention may include other active ingredients. These active ingredients may be, in particular, those with an effect similar or supplementary to the effect mediated by lipoic acid or glutamine. Thus, it may be advantageous to administer in addition to the combination of the invention antioxidants, polycosanols, S-adenosylmethionine, choline, flavonoids, lignans, isoflavones. Vitamins, cofactors, trace elements, especially Cr, Se, Mn, Zn, minerals, amino acids and other essential nutrients may also be expedient. For practical reasons, in many cases fatty acids, where appropriate in the form of glycerides, will also be included. Polyunsaturated fatty acids, especially ω-3- and ω-6-PUFA, e.g. arachidonic acid and, in particular, docosahexaenoic acid and/or eicosapentaenoic acid; phospholipids, especially phosphatidylcholine, phosphatidylserine and phosphatidylethanolamine; antioxidants, especially vitamin E, in particular as tocopherol, tocopherol acetate or tocopheryl succinate, and vitamin C, in particular as ascorbic acid, Na, Ca or K ascorbate, or ascorbyl-6-palmitate, flavonoids, isoflavones, lignans, tocotrienols, etc. are preferably administered together with the lipoic acid and glutamine components.

A particular embodiment of the present invention is based on the combination of lipoic acid with an R enantiomeric excess, in particular as trometamol salt, and glutamine with an S enantiomeric excess, in particular in the form of a dipeptide.

The invention encompasses within the scope of therapeutic uses, of a nutritional supplementation, of a dietary nutritional strategy or in the area of fortified foods (functional foods) a treatment of individuals.

In the area of nutritional supplementation, the intake ensured by the normal diet is supplemented by an active ingredient combination of the invention. In this sense, the active ingredient combination of the invention is also to be regarded as a nutrient combination. The purpose of this nutritional supplementation may be to compensate for corresponding dietary deficiencies or to ensure an intake of these active ingredients which is above the amount ensured with the normal diet. The active ingredient combination of the invention can in particular also be used within the framework of a nutrition therapy, e.g. in parenteral or enteral nutrition. Thus, the use according to the invention for nutritional supplementation also serves physiological dietary purposes, in particular the treatment of corresponding deficiency symptoms and alteration of particular states of an individual, which can be respectively compensated and brought about with a nutritional supplementary intake of the active ingredient combination of the invention. The deficiency symptoms and alterable states include the disorders which can be treated, and the effects which can be achieved, according to the invention, which are listed below. In this sense, it is one aspect to prevent a glutathione deficiency or compensate for a deficiency which already exists.

The use according to the invention for therapeutic purposes relates in particular to the treatment of disorders associated with a glutathione deficiency. Accordingly, one aspect of the use according to the invention is directed at stabilizing cellular metabolism. By stabilizing cellular metabolism is meant according to the invention a time delay or an at least partial reverse of a change in one or more metabolic parameters which causes metabolic disturbances.

A particular aspect relates to cellular glutathione metabolism. Metabolic parameters which should be particularly mentioned under this aspect are intracellular concentrations of reduced (GSH) and oxidized glutathione (GSSG) and the ratio of GSH to GSSG.

In a particular embodiment of the present invention, the stabilization of cellular glutathione metabolism relates to an increase in the intracellular concentration of GSH and/or GSSG, advantageously in particular of GSH, and especially an increase in the GSH/GSSG concentration ratio. In this connection, the ratio of reduced glutathione (GSH) to oxidized glutathione (GSSG) is advantageously at least about 500.

Particular embodiments of the use according to the invention relate mainly to glutathione metabolism in hepatocytes or spleen cells and advantageously intestinal cells, especially small intestinal cells, and in this case in particular cells of the GALT (gut-associated lymphoid tissue), which include in particular cells of Peyer's plaques. The active ingredient combination of the invention can thus be used for the treatment of a depletion of intracellular glutathione, especially associated with ischemia reperfusion (reperfusion syndrome); myocardial infarction; respiratory failure; cancer; diabetes, e.g. in type 2 diabetes to increase insulin sensitivity (De Mattia G, et al., Metabolism 47: 993-997; 1998); liver disorders (depletion of hepatic glutathione), e.g. damage to hepatocytes and endothelial cells through chemotherapy, especially after bone marrow transplantation (Brown S A, et al., Bone Marrow Transplantation 22:281-284; 1998); Alzheimer's; inflammatory bowel disorders (Sido B, et al., Gut 42:485-492; 1998; Miralles-Barrachina O, et al., Clin Nutrition 18:313-317; 1999), in clinical protein catabolism, e.g. after operations, trauma, burns, in sepsis and/or in intensive care patients; to potentiate the vasodilating effect of NO (Kugiyama K, et al., Circulation 97: 2299-2301; 1998); in an anemia caused by uremia after hemodialysis (Usberti M, et al., Journal of Nephrology 10: 261-265; 1997); in cystic fibrosis, especially for reduced release of toxic oxidizing compounds (Roum J H, et al., Journal of Applied Physiology 87: 438-443; 1999); in HIV-infected patients (Staal FJT, et al., Aids Research and Human Retroviruses 12:1373-1381; 1996), especially to reduce the HIV virus in various tissues (Palamara AT, et al., Aids Research and Human Retroviruses 12:1373-1381, 1996).

A further particular aspect relates to the use of the active ingredient combination of the invention for modulating spleen immunology and advantageously intestinal immunology. This includes in particular an increase in the number of lymphocytes in the GALT (gut-associated lymphoid tissue), e.g. the lamina propria, the intestinal epithelium, the mesenteric lymph nodes and, in particular, Peyer's plaques. The effects of the invention associated therewith include an enhancement of lymphocyte proliferation; of the differentiation of B cells into antibody-producing cells; of immunoglobulin production, of T-cell activation; of monocyte and macrophage function.

Preferred embodiments of the present invention are directed at the treatment of one of the following disorders: sepsis; ARDS, Crohn's disease; colitis, short-bowel syndrome; intestinal transplantation; diarrhea; enteritis; infectious diseases; pancreatitis; cirrhosis of the liver; AIDS.

The importance of the treatment according to the invention increases in adults with increasing age. The treatment has particular advantages in the group of those over 40 years of age and especially of those over 50 years of age. This also applies to the burn-out syndrome and where physical effort is increased, e.g. in athletes.

Disorders and diseases to be treated according to the invention are usually characterized by a progressive development, e.g. the states described above change over the course of time, usually with an increase in severity, and it is possible where appropriate for states to interchange or for other states to be added to previously existing states. Thus, preventive treatment represents a particularly valuable aspect of the treatment according to the invention.

It is possible via the treatment according to the invention to treat a plurality of signs, symptoms and/or dysfunctions associated with the aforementioned disorders and states. These include, for example, infections, influenza infections, convalescence, poor general condition, increased physical effort, stress.

One aspect of the treatment in the sense according to the invention relates to the treatment of acute and chronic disorders, states, signs, symptoms and/or dysfunctions; one purpose of this treatment is to eliminate the disorders, regulate the states, or alleviate the signs, symptoms and/or dysfunctions. A further aspect relates to a preventive treatment (prophylaxis), in particular in relation to the aforementioned disorders with an oxidative cause; one purpose of this treatment is to avert the occurrence of the disorders, states, signs, symptoms and/or dysfunctions, which also includes the postponement of the occurrence. The treatment may aim at being symptomatic, for example as symptom suppression. It may take place short-term, aim at being medium-term or else comprise a long-term treatment, for example as part of a maintenance therapy. The treatment may also take place as a course of treatment, for example in the form of continuous treatments for several days or several weeks alternating with breaks in intake.

The use according to the invention of the described active ingredients comprises a method within the scope of the treatment. This entails the individual to be treated, preferably a mammal, in particular a human or agricultural or domestic animal, being given an effective amount of lipoic acid component and an effective amount of glutamine component, usually formulated in accordance with the practice of pharmacy, veterinary medicine or food technology. Whether such a treatment is indicated, and the form it is to take, depend on the individual case and may be subject both to specialist medical (usually objective diagnosis) and nonspecialist assessment (usually self-diagnosis) which may take account of the signs, symptoms and/or dysfunctions present, the risks of developing certain signs, symptoms and/or dysfunctions, and other factors.

The treatment usually takes place by single, multiple or continuous daily administration, where appropriate together or alternately with other active ingredients or active ingredient-containing products. It is important according to the invention that more glutamine than lipoic acid is administered to the individual to be treated. A molar ratio of glutamine to lipoic acid of at least about 3 is ordinarily used, preferably of at least about 6, in particular of at least about 10 and particularly advantageously of at least about 25. For example, a daily dose administered to an individual to be treated and weighing about 70 kg is about 1 mg to 5 g, preferably about 10 mg to 1 g, of lipoic acid; and about 1 g to 200 g, preferably about 5 g to 100 g, of glutamine on oral administration, and preferably about 5 mg to 1 g of lipoic acid, and about 5 g to 100 g of glutamine, on parenteral administration.

The amounts and proportions of active ingredients are based on the active ingredient, i.e. lipoic acid and glutamine, so that an appropriate conversion is necessary for salts and derivatives.

The invention also relates to the production of compositions for the treatment of an individual, preferably a mammal, in particular a human, and also an agricultural or domestic animal.

The present invention therefore also relates to compositions comprising

i) at least one lipoic acid, physiologically acceptable derivatives or salts thereof;

ii) at least one glutamine, physiologically acceptable derivatives or salts thereof; and, where appropriate, at least one other active ingredient and a formulation base.

Compositions of the invention are therefore based on an active ingredient combination and, where appropriate, a formulation base.

The compositions include, in particular, pharmaceutical compositions, nutritional supplements and food products, in particular functional or dietary foods. The food products of the invention have, besides a predominantly nutritional-related function, additionally an active ingredient-related function which relates in particular to the active ingredient combination of the invention. They are therefore referred to as functional or dietetic foods or nutrients. Nutritional supplements have to supplement the daily diet with the active ingredient combination of the invention, with the nutrition-related function of the nutritional supplement on its own becoming of less importance.

The active ingredient combination for the purposes of the invention comprises as active ingredient component i) at least one lipoic acid, a physiologically acceptable derivative or salt thereof. Mixtures of these forms are possible, but will be considered only in certain cases. In a particular embodiment, the active ingredient component i) consists of lipoic acid, preferably at least 90% by weight, and in particular at least 99% by weight, of the (R) enantiomer, where the percent by weight data are based on the total weight of the active ingredient component i).

The active ingredient combination further comprises for the purposes of the invention as active ingredient component ii) at least one glutamine, a physiologically acceptable derivative or salt thereof. The active ingredients listed above as particular glutamines are preferred, especially the dipeptides.

The active ingredient combination may further comprise for the purposes of the invention as active ingredient component iii) further active ingredients, for example the active ingredients mentioned in this connection above.

The proportion of the active ingredient combination in the formulation is larger than a proportion present where appropriate in natural sources, especially foods. In this sense, the compositions of the invention are enriched in relation to the active ingredient combination in particular compared with foods. The proportion of the active ingredient combination of i) and ii) in the formulation is preferably greater than about 0.5% by weight, advantageously greater than about 1% by weight and in particular greater than about 2% by weight. In particular, the proportion of i) is more than 0.01% by weight, preferably more than 0.05% by weight, and in particular more than 1% by weight; the proportion of ii) is more than 1% by weight, preferably more than 5% by weight, and in particular more than 10% by weight. In the case of a pharmaceutical composition, the proportion of i) and ii) is usually about 1 to 60% by weight, preferably about 5 to 35% by weight, and in particular about 10 to 30% by weight, and in the case of a nutritional supplement and especially in food products is where appropriate correspondingly lower if the formulation is administered in larger amounts.

Unless otherwise indicated, data in percent by weight are based on the total weight of the formulation.

The formulation base for formulations of the invention comprises physiologically acceptable excipients. Physiologically acceptable excipients are those known to be usable in the sectors of pharmacy, food technology and adjacent areas, in particular the excipients listed in relevant pharmacopeias (e.g. DAB, Ph. Eur., BP, NF), and other excipients whose properties do not stand in the way of physiological use. Excipients for the purpose of the invention may also have a nutritional value and are therefore generally used as food component. They may also include nutrients, especially essential nutrients.

Suitable excipients may be: wetting agents; emulsifying and suspending agents; preservatives; antioxidants; antiirritants; chelating agents; tablet coating aids; emulsion stabilizers; film formers; gel formers; odor-masking agents; masking flavors; resins; hydrocolloids; solvents; solubilizers; neutralizers; permeation promoters; pigments; quaternary ammonium compounds; refatting and superfatting agents; ointment, cream or oil bases; silicone derivatives; spreading aids; stabilizers; sterilants; suppository bases; tablet excipients such as binders, fillers, lubricants, disintegrants or coatings; propellants; desiccaints; opacifiers; thickeners; waxes; plasticizers; white oils. An arrangement concerning this is based on specialist knowledge as described, for example, in Fiedler, H. P., Lexikon der Hilfsstoffe für Pharmazie, Kosmetik und angrenzende Gebiete, 4th edition, Aulendorf: ECV-Editio-Cantor-Verlag, 1996.

Food components usually comprise one or more amino acids, carbohydrates or fats and are suitable for the human and/or animal diet. They comprise individual components, frequently vegetable but also animal products, especially sucrose, where appropriate in the form of syrups, fruit preparations such as fruit juices, nectar, fruit pulps, purees or dried fruit, for example apple juice, grapefruit juice, orange juice, apple puree, tomato sauce, tomato juice, tomato puree; cereal products such as wheat flour, rye flour, oat flour, corn flour, barley flour, spelt flour, corn syrup and starches from said cereals; dairy products such as milk protein, whey, yoghurt, lecithin and lactose.

Essential nutrients include, in particular, vitamins, provitamins, minerals, trace elements, amino acids and fatty acids. Essential amino acids which may be mentioned are isoleucine, leucine, lysine, methionine, phenylalanine, threonine, tryptophan and valine. They also include semiessential amino acids which must be given, for example, in periods of growth or deficiency states, such as arginine, histidine, cysteine and tyrosine. Trace elements which may be mentioned are: essential trace elements and minerals which have been proved to be necessary for humans and deficiency of which leads to manifestation of clinical symptoms: iron, copper, zinc, chromium, selenium, calcium, magnesium, sodium, potassium, manganese, chlorine, cobalt, molybdenum, iodine, silicon, fluorine, phosphorus. Likewise elements whose function in humans is as yet inadequately verified: tin, nickel, vanadium, arsenic, lithium, lead, boron. Fatty acids essential for humans which may be mentioned are: linoleic acid and linolenic acid, ARA (arachidonic acid) and DHA (docosahexaenoic acid) for infants and possibly EPA (eicosapentaneoic acid) and DHA also for adults. A comprehensive list of vitamins is to be found in “Referenzwerte für die Nährstoffzufuhr”, 1st edition, Umschau Braus Verlag, Frankfurt am Main, 2000, edited by the Deutsche Gesellschaft für Ernährung.

The total of active ingredient component and formulation base is usually 100% by weight.

Examples of suitable formulations for nutritional supplementation are capsules, tablets, pills, powder sachets, liquid ampuls and bottles with integral droppers, as well as the drug forms mentioned below.

Examples of suitable pharmaceutical formulations are solid drug forms such as oral powders, dusting powders, granules, tablets, especially film-coated tablets, pastilles, sachets, cachets, sugar-coated tablets, capsules such as hard and soft gelatin capsules, suppositories or vaginal drug forms, semisolid drug forms such as ointments, creams, hydrogels, pastes or plasters, and liquid drug forms which are used in the present case in particular for parenteral or enteral alimentation, such as solutions, emulsions, especially oil-in-water emulsions, suspensions, preparations for injection and infusion. It is also possible to use implanted delivery devices for administering active ingredients of the invention. Liposomes or microspheres may also be used.

Foodstuff formulations usually have the normal form and are preferably marketed in the form of infant food, breakfast products, especially in the form of mueslis or bars, sports beverages, complete meals, especially in the framework of completely balanced diets, dietary products such as diet beverages, diet meals and diet bars.

The formulations are usually administered by the enteral or parenteral and preferably by the oral route. A particular form of enteral administration relates to administration by a tube (tube feeding). Parenteral administration relates in particular to central or peripheral venous administration.

For producing the compositions, the active ingredients are usually mixed or diluted with a suitable excipient. Excipients may be solid, semisolid or liquid materials serving as vehicle, carrier or medium for the active ingredient. Admixture of other excipients takes place, if necessary, in a manner known per se. It is possible to carry out shaping steps, where appropriate in conjunction with mixing processes, e.g. a granulation, compression and the like.

The active ingredient components can, in particular, be formulated together. However, they may also be initially processed separately and subsequently combined in a compartmented, e.g. multilayer, drug form. It is possible in this way to take account of possible active ingredient incompatibilities and different active ingredient properties such as bioavailability, stability, solubility and the like. Gastroresistant formulations are suitable for enteral and especially oral administration.

The present invention further relates to compositions in the form of a commercial pack having at least one composition based on

i) at least one lipoic acid, physiologically acceptable derivatives and/or salts thereof, and/or

ii) at least one glutamine, physiologically acceptable derivatives and/or salts thereof,

where appropriate together with instructions for the combined use of lipoic acids, physiologically acceptable derivatives or salts thereof and glutamines, physiologically acceptable derivatives or salts thereof.

One embodiment of this aspect of the invention relates to commercial packs having at least one, in particular pharmaceutical, composition of the type described above with an active ingredient combination of the invention. This embodiment also encompasses commercial packs having a plurality of combination products in diverse dosages or formulations. Commercial packs of this embodiment accordingly comprise active ingredient components i) and ii) formulated together.

Another embodiment relates to commercial packs having two or more, in particular pharmaceutical, compositions which are spatially separated from one another and of which at least two compositions comprise different active ingredients. These compositions may be, in particular, single-component products, i.e. especially those with active ingredient component i) or ii). In these cases, the commercial pack contains instructions in the sense of the invention for the combined use of the compositions comprising i) and ii). Commercial packs of this embodiment accordingly comprise active ingredient components i) and/or ii) formulated separately, i.e. in the form of at least two spatially separate compositions.

Another embodiment relates to commercial packs having at least one, in particular pharmaceutical, composition based on

i) at least one lipoic acid, physiologically acceptable derivatives or salts thereof; or

ii) at least one glutamine, physiologically acceptable derivatives or salts thereof.

These take the form of single-component products. In these cases, the commercial pack contains instructions in the sense of the invention for the therapeutic use of the composition in combination with the other active ingredients which form the active ingredient combination of the invention but are not part of the commercial pack, in the form of at least one other composition. Commercial packs of this embodiment accordingly comprise part of the active ingredient combination of the invention. The part which is not contained is included as intended as part of the enclosed instructions.

It is self-evident that commercial packs of the invention may also comprise other products, especially active ingredient-containing formulations, and comprehensive instructions also going beyond the aforementioned contents.

The present invention is explained in detail by means of the following examples without being restricted thereto.

The drawings show for a number of test mixtures to which L-glutamine (GLN), R-lipoic acid (LA) or combinations thereof had been added:

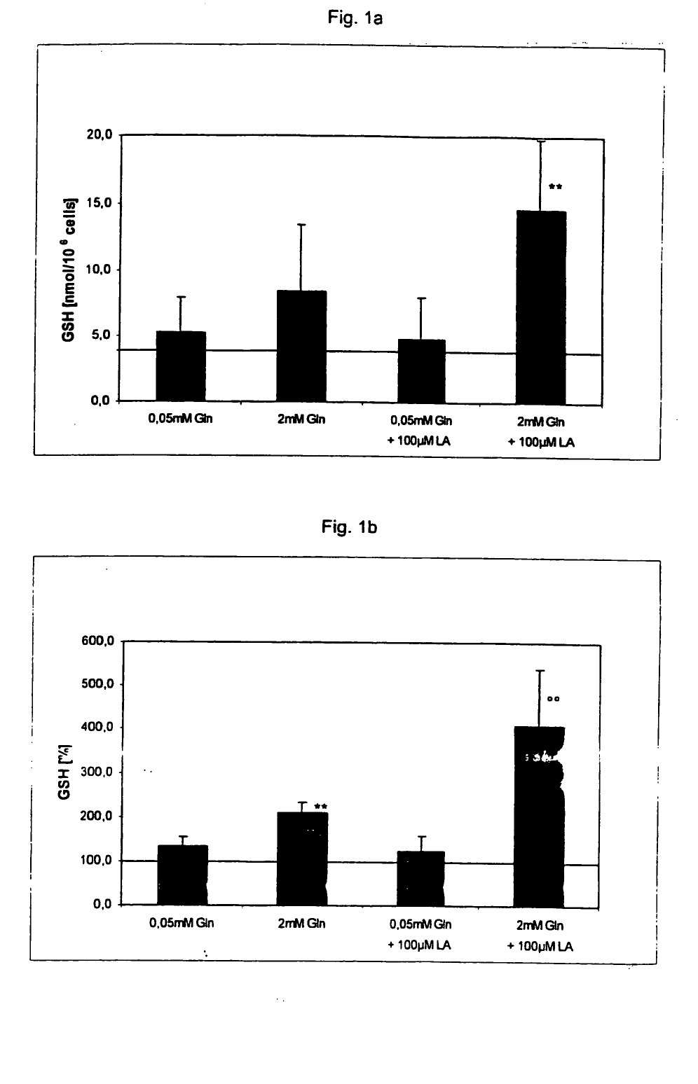

FIG. 1 the concentration, plotted in the form of a bar diagram, absolute (1a) and based on the initial concentration before addition of glutamine (Gln) and/or lipoic acid (LA) (1b), of reduced glutathione (GSH) in human Jurkat T cells;

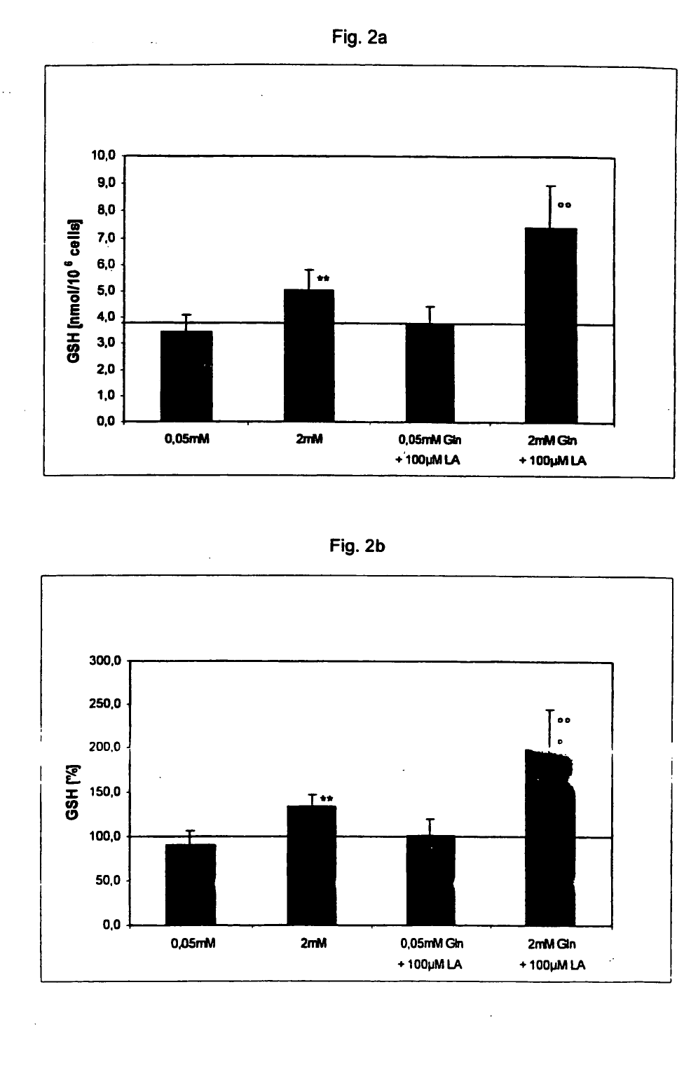

FIG. 2 the concentration, plotted in the form of a bar diagram, absolute (2a) and based on the initial concentration before addition of glutamine (Gln) and/or lipoic acid (2b), of reduced glutathione (GSH) in human U937 cells.

### EXAMPLE 1

Pharmaceutical Compositions

a) Soft-gelatin capsule with lipoic acid and glutamine (lipoic acid 50 mg+glutamine 1 g)

| | |
|---|---|
| Lipoic acid | 50 mg |
| Glutamine | 1 g |
| D/L-alpha-Tocopherol | 60 mg |

### EXAMPLE 2

Functional Food

a) Bar with lipoic acid and glutamine (400 mg of lipoic acid+glutamine 4 g/ar (60 g))

| | |
|---|---|
| Lipoic acid | 400 mg |
| Glutamine | 4 g |
| D/L-alpha-Tocopherol | 150 mg |
| Syrup from | |
| fructose | 4.2 g |
| glucose | 12 g |
| caramelized sugar | 3 g |
| glycerol | 3 g |
| Lecithin | 125 mg |
| Hydrogenated vegetable oil | 1.2 g |
| Roasted oat flakes | 17.975 g |
| Puffed rice | 7 g |
| Roasted and chopped almonds | 5.6 g |
| Coconut flakes | 4 g |

### EXAMPLE 3

### Parenteral Alimentation

3.1. TPN plus glutamine and lipoic acid:

Composition as Glamin (Fresenius-Kabi) plus 400 mg of lipoic acid

3.2. Parenteral supplement: glutamine and lipoic acid Composition as Dipeptamin (Fresenius-Kabi) plus 400 mg of lipoic acid

### EXAMPLE 4

### Enteral Alimentation

4.1. Total enteral alimentation plus glutamine and lipoic acid Content per 100 ml:

Nitrogen source

| | |
|---|---|
| (Protein + oligoproteins + | 6 g |
| glutamine dipeptides) | |
| Fat source | 2.5 g |
| (vegetable oils, medium | |
| chain-length triglycerides, fish oil: | |
| ω-3:ω-6 1:3.5) | |
| Carbohydrates | 12 g |
| (Maltodextrins, polysccharides, sucrose) | |
| Dietary fiber | 1.5 g |
| (soluble + insoluble) | |
| Lipoic acid | 400 mg |
| Vitamin C | 1000 mg |
| Vitamin E | 200 mg |
| Selenium | 20 μg |
| Minerals | complying with RDA |
| (sodium, potassium, chloride, | |
| potassium, phosphate, magnesium) | |
| Trace elements | complying with RDA |
| (iron, copper, manganese, iodide, | |
| fluoride, molybdenum) | |
| Other vitamins | complying with RDA |
| 4.2. Enteral supplement: glutamine and lipoic acid | |
| Glutamine (alanylglutamine, glycylglutamine) | 25 g |
| Vitamin E | 200 mg |
| Lipoic acid | 400 mg |
| Vitamin C | 1000 mg |
| Vitamin E | 200 mg |
| Selenium | 200 μg |

### EXAMPLE 5

Biological Effect

Increase in intracellular glutathione levels

Human Jurkat T cells and U937 cells, a human myelomonocyclic cell line, were used for the experiments. Jurkat and U937 cells were cultivated in RPMI 1640 medium (Bio Whittaker, Belgium)—supplemented with 10% heat-inactivated fetal calf serum (FCS; Linaris, Germany), 1% penicillin/streptomycin (Life Technologies, Scotland) and 2 mM L-glutamine (Sigma, Mont.)—under standard conditions (95% humidity, 5% CO₂ and 37° C.).

After harvesting, the cells were washed 2× with PBS, resuspended in standard medium (RPMI 1640 with 10% FCS, 1% streptomycin/penicillin, without L-glutamine) in a concentration of 2×10⁵ cells/ml and transferred into 25 cm³ culture bottles. The cells were treated with and without addition of 2 mM glutamine and in each case with or without 100 μM α-lipoic acid over a period of 4, 8, 24 or 48 h.

After the respective treatment period had elapsed, the cells were harvested and washed 2× with PBS. 2×10⁶ cells were resuspended in sulfosalicylic acid (6.5% in distilled water; Marck, Germany) and incubated on ice for 15 min. The supernatant after pelleting of the cells (5 000 g, 10 min) was aspirated off and stored at −20° C. until glutathione was determined by HPLC.

As is evident from FIGS. 1 and 2, addition of 0.05 mM glutamine to the culture medium caused no significant change in the concentration of reduced glutathione (GSH). This also applies on simultaneous addition of 100 μM lipoic acid. However, if 2 mM glutamine are added to the culture medium, not only are there significant increases in the concentrations of reduced glutathione, also simultaneous addition of lipoic acid in the previously inactive concentration of 100 μM causes a drastic rise in the GSH concentrations.

These results prove that synergistic effects on the glutathione metabolism of the cell types used here can be achieved through combined administration of glutamine and lipoic acid.

1. The use of an active ingredient combination of
i) at least one lipoic acid, a physiologically acceptable derivative and/or salt thereof; and
ii) at least one glutamine, a physiologically acceptable derivative and/or salt thereof,
for producing a composition for parenteral nutrition therapy.

2. The use of an active ingredient combination of
i) at least one lipoic acid, a physiologically acceptable derivative and/or salt thereof; and
ii) at least one glutamine, a physiologically acceptable derivative and/or salt thereof,
for producing a composition for enteral nutrition therapy.

3. The use as claimed in claims 1 or 2, where the composition is used to supplement the nutrition therapy.

4. The use of an active ingredient combination of
i) at least one lipoic acid, a physiologically acceptable derivative and/or salt thereof; and
ii) at least one glutamine, a physiologically acceptable derivative and/or salt thereof,
for producing a composition for stabilizing the cellular glutathione metabolism of intestinal cells.

5. The use as claimed in claim 4 for increasing intracellular GSH concentrations.

6. The use of an acitve ingredient combination of
i) at least one lipoic acid, a physiologically acceptable derivative and/or salt thereof; and
ii) at least one glutamine, a physiologically acceptable derivative and/or salt thereof,
for producing a composition for the enteral or parenteral treatment of disorders associated with a glutathione deficiency of intestinal cells.

7. The use as claimed in anyone of claims 4 to 6, where the intestinal cells are cells of the GALT.

8. The use as claimed in claim 7, where the cells of the GALT are cells of Peyer's plaques.

9. The use of an acitve ingredient combination of
i) at least one lipoic acid, a physiologically acceptable derivative and/or salt thereof; and
ii) at least one glutamine, a physiologically acceptable derivative and/or salt thereof,
for producing a composition for strengthening the intestinal immune system.

10. The use as claimed in claim 9, where the strengthening of the intestinal immune system is an increase in the number of lymphocytes in the GALT.

11. The use as claimed in claim 9, where the strengthening of the intestinal immune system is an increase of lymphocytes in Peyer's plaques.
